(19) United States
(12) Patent Application Publication
(10) Pub. No.: US 2009/0028896 A1
(43) Pub. Date: Jan. 29, 2009

(54) COMPOSITIONS AND METHOD FOR WEIGHT LOSS

(75) Inventor: Yoshinori TAKEDA, Columbia, MD (US)

Correspondence Address: MCDERMOTT WILL & EMERY LLP, 600 13TH STREET, N.W., WASHINGTON, DC, 20005-3096, US

(73) Assignee: PRIMAVERA BIOSCIENCES, INC., Columbia, MD (US)

(21) Appl. No.: 12/240,236

(22) Filed: Sep. 29, 2008

Related U.S. Application Data
(62) Division of application No. 10/831,353, filed on Apr. 26, 2004.

(51) Int. Cl. A61K 36/06; A61P 3/00

(52) U.S. Cl. 424/195.15

(57) ABSTRACT

Dietary supplements and methods for management and control of body weight comprising at least one liver protecting agent and at least one mushroom in the form of powder, extract, or combinations thereof.

### FIELD OF INVENTION

The present invention relates to compositions and methods for management and control of body weight. The present invention also relates to compositions and methods for reducing body weight and treating obesity. In particular, the invention relates to dietary supplement compositions and methods for weight reduction comprising at least one liver protecting agent and at least one mushroom.

### BACKGROUND OF THE INVENTION

Obesity rates are soaring in the United States and many other countries. Fifty-eight percent of American adults are overweight, and the recent government figures show obesity on the rise in both sexes and in every age group. Existing therapies for overweight and obese people are to establish a negative energy balance. This may be accomplished by reduction of calorie intake, such as having reduced-calorie diets that manage fat and carbohydrate, or by increasing calorie expenditure with physical activities and exercises. However, far too often individuals abandon reduced-calorie diet regimes before they reach their goals because they struggle against ingrained eating habits, feelings of hunger, emotional pressures, and other discouragement. Similarly, many individuals also fail to adhere to physical activity regimes over a long period.

In recent years much attention has been focused on developing pharmaceutical solutions to the obesity problem. One such pharmaceutical approach includes ingestion of sympathomimetic drugs which stimulate thermogenesis (i.e., to increase the metabolic rate). Known thermogenic drugs include ephedrine, phenylpropanolamine, and caffeine. Another approach, like that of XENICAL® (HLR Technology Corporation), is to locally inhibit gastro-intestinal (GI) lipases, thereby blocking the absorption of dietary fat.

Other drugs to reduce, maintain and control body weight include amphetamine-like agents that suppress appetite by acting on the hypothalamic center. Nazindol and derivatives of phenethylamine are known to act on the noradrenergic neurotransmitter while fenfluramine, tryptophan, fluoxetine and sertraline are known to act on the serotonin neurotransmitter. MERIDIA® (Abbott Laboratories Corporation) works by affecting appetite control centers in the brain via the norepinephrine, serotonin and dopamine reuptake inhibition, and thereby reducing food intake. All of these drugs, however, have undesirable side effects and their use is not recommended for extended periods.

Natural supplements are also used in weight management: for example, combinations and formulations of: fat burners (e.g., chromium picolinate and L-carnitine), lipogenesis inhibitors (e.g., Garcinia cambogia), appetite suppressants (e.g., DL-phenylalanine and L-glutamine), bulking agents to instill a feeling of satiety (e.g., psyllium, guar gum and glucomannan) and many others. However, although many claims have been made, via some clinical trials, that they are effective in weight control, none of these therapies and/or formulas have adequately proved to become universally accepted methods for safe and sustained weight reduction.

The present invention focuses on normalizing the fat and carbohydrate metabolisms in the liver, thereby providing a unique solution for obesity and weight control problems.

When people start losing their body weight, they frequently observe that their weight loss slows after an initial loss, probably because the body, after the initial weight loss, reduces metabolic rates and fat breakdown. Many dieters also observe that they have stronger sensations of hunger after they have lost some body weight. Recent research has shown that increased hunger sensation is due to the elevated level of hunger hormone ghrelin. Slowed metabolisms and elevated ghrelin are believed to be the major causes resulting in many dieters experiencing a weight loss plateau at which, after initial weight loss, a further weight loss is inhibited and/or slowed for a period of time. The compositions and methods of the present invention appear to eliminate a weight loss plateau by suppressing hunger and not slowing fat breakdown, and thus confer sustained weight reduction.

The body senses when to eat through various chemical messengers that stimulate or suppress hunger and appetite. The hypothalamus keeps track of the body's nutritional intake by monitoring chemical signals from various organs and tissues, including stomach, intestinal tract, pancreas and fat cells. A major center that controls feeding is the area of the hypothalamus called the arcuate nucleus. Within the nucleus, one group of nerve cells, the NPY/AgRP neurons, stimulates appetite and food intake, and another group, the POMC/CART neurons, suppresses it. The two groups of nerve cells communicate with each other. The hormones leptin (made by adipose cells) and insulin (made by pancreas) act on both groups of nerve cells and reduce food intake. On the other hand, the recently discovered hormone ghrelin (chiefly made by stomach) acts at the NPY/AgRP nerve cells, stimulates feelings of hunger and increases food intake. Ghrelin secretion follows a daily pattern, peaking just before each meal and falling afterward. Ghrelin also acts on other tissues to slow metabolism and fat burning. Without intending to be bound by theory, it is believed that the compositions and methods of the present invention suppress hunger by lowering the ghrelin level to maintain burning fats even after weight loss.

### SUMMARY OF THE INVENTION

The present invention provides dietary supplement compositions and methods that are useful for the management of body weight. In particular, the present invention provides safe and effective dietary supplements that provide sustained weight reduction by effectively suppressing hunger and helping to efficiently burn body fats. Generally, the dietary supplement compositions comprise effective amounts of at least one liver protecting agent and at least one mushroom powder, extract, or derivative thereof, and the methods involve administering together or substantially together at least one liver protecting agent and at least one mushroom powder, extract or derivative thereof to achieve weight management. The present invention includes neither controversial stimulants, such as ephedrine and caffeine, nor pharmaceutical drugs.

The present invention also provides safe and effective compositions and methods that are useful for the treatment of high cholesterol, diabetes, hypertension, and liver problems induced by alcohol, drugs, or chemicals. The administration of the compositions of the present invention can promote the reduction of high cholesterol, promote the reduction of high blood glucose in diabetics, promote the reduction of high blood pressure; and promote healing of the liver damaged by alcohol, drugs, or chemicals

In one aspect of the invention there are safe and effective dietary supplements for sustained weight reduction comprising at least one liver protecting agent selected from the group consisting of Andrographis, artichoke, Artemisia, astragalus, barberry, boldo, bupleurum, dandelion, dong quai, fo-ti, fringe tree, fumitory, gotu kola, guggul, kudzu, licorice, lycium, milk thistle, neem, Phyllanthus, Picrorrhiza, prickly pear, rehmannia, skullcap, schisandra, tumeric and combinations thereof and at least one mushroom selected from a genus of the group consisting of Agaricus, Auricularia, Cordyceps, Coriolus, Ganoderma, Grifola, Hericuim, Lentinus, Pleurotus, Polyporus, Poria, Trametes, Tremella, and combinations thereof. In a preferred embodiment, the compositions of the present invention suppress hunger. In another preferred embodiment, the compositions increase the efficiency of burning fat. In still another preferred embodiment, the dietary supplements inhibit fat absorption. In still another preferred embodiment, the dietary supplements provide sustained weight reduction substantially free from a weight loss plateau. In yet another preferred embodiment, the dietary supplements help to reduce cholesterol levels. In yet another preferred embodiment, the dietary supplements help to reduce blood glucose levels with diabetic people. In yet another preferred embodiment, the compositions help to lower blood pressure with people with hypertension. In yet another preferred embodiment, the compositions help to heal a liver damaged by alcohol, drugs and chemicals. The at least one liver protecting agent and the at least one mushroom are preferably in the powder or extract form. It is also preferable that the dietary supplements for sustained weight loss are in an oral dosage form, more preferably, a solid dosage form, such as a tablet or capsule.

In another aspect of the present invention there are safe and effective methods for sustained weight reduction comprising administering to a subject in need thereof a dietary supplement composition comprising at least one liver protecting agent and one or more mushroom. Preferably the dietary supplement composition is administered and/or ingested in conjunction with any method to attain a negative energy balance. More preferably, negative energy balance is achieved through exercise and/or calorie restriction.

The liver controls the storage and utilization of energy sources: it controls the conversion of glucose to glycogen and vice versa and also controls the fat metabolism. Accordingly, the present invention provides safe and effective compositions and methods that help rejuvenate the fatty liver and normalize fat and carbohydrate metabolisms. By doing so, the present invention provides safe and effective dietary supplement compositions and methods that are useful for suppressing hunger and efficiently burning body fats for the easier management and control of body weight. The present invention also inhibits fat absorption in the digestive tract. Furthermore, compositions of the present invention when combined with any method to attain a negative energy balance, increase the efficiency of burning of body fats more than the composition alone, to achieve sustained reduction of body weight.

At least one of the above embodiments and advantages may be realized and attained by means of the instrumentalities and combinations particularly recited in the appended claims and/or supported by this written description. Additional embodiments and attendant advantages of the present invention will be set forth, in part, in the description that follows, or may be learned from practicing or using the present invention. It is to be understood that the foregoing general description and the following detailed description are exemplary and explanatory and are not to be viewed as being restrictive of the invention, as claimed.

### BRIEF DESCRIPTION OF THE DRAWINGS

FIG. 1. shows the change in, and/or maintenance of, weight (lbs.) over time (days) of the individual in Example 4 (Case 1).

FIG. 2. shows the change in, and/or maintenance of, weight (lbs.) over time (days) of the individual in Example 5 (Case 2).

FIG. 3. shows the change in, and/or maintenance of, weight (lbs.) over time (days) of the individual in Example 6 (Case 3).

FIG. 4. shows the suppression of weight gain over time in mice (Group 2) which were fed a high fat diet with a 45% fat content and a dietary supplement of: 2.5% shisandra, 2.5% lycium, 7.5% maitake, and 7.5% reishi (all in powder). The control group mice of Group 1 which were fed a high fat diet alone without the dietary supplement fed to Group 2 gained weight. The data are presented as the average weights of five mice in each group with a standard deviation.

FIG. 5. shows a typical mouse after 21 days from each of Group 1 and Group 2 in the experiment described in Example 7, date for which is shown in FIG. 4. The observably smaller mouse was fed the high fat diet in combination with the dietary supplement; the larger mouse was fed the high fat diet alone.

### DETAILED DESCRIPTION OF THE INVENTION

In a preferred embodiment, the present invention provides a novel, safe and effective dietary supplement composition for sustained weight reduction comprising at least one liver protecting agent and at least one mushroom each in the form of a powder, extract, or derivative thereof. In a further preferred embodiment, the present invention is directed to a safe and effective dietary supplement composition for sustained weight reduction comprising at least one liver protecting agent, at least one mushroom, and one or more nutraceutically or pharmaceutically acceptable carrier, wherein the composition suppresses hunger and metabolizes fat for sustained weight reduction substantially free of a weight loss plateau. Preferably, the embodiments of the present invention are combined with a method and/or regime that achieves a negative energy balance.

As used herein, the term “appetite” refers to the instinctive desires necessary to keep up organic life; especially, the desire to eat or an inherent craving to eat.

As used herein, the term “appetite suppressant” refers to the substance that suppresses appetite.

As used herein, the term “derivative” refers to a chemical substance derived from and/or related structurally to an extract, powder, or any other forms.

As used herein, the term “hunger” refers to an uneasy sensation occasioned by the lack of food.

As used herein, the term “hunger is suppressed” refers to a state in which the conditions of hunger are restrained, inhibited or suppressed, or the intensity of feelings of hunger is subdued or suppressed to varying degrees.

As used herein, the term “liver protecting agent” refers to a substance that confers a beneficial advantage in the protection of and/or treatment of the liver. Exemplary liver protecting agents contemplated by the present invention include Andrographis, artichoke, Artemisia, astragalus, barberry, boldo, bupleurum, dandelion, dong quai, fo-ti, fringe tree, fumitory, gotu kola, guggul, kudzu, licorice, lycium, milk thistle, neem, Phyllanthus, Picrorrhiza, prickly pear, rehmannia, skullcap, schisandra, and tumeric.

As used herein, the term “mushroom” refers to a mushroom selected from a genus of the group consisting of Agaricus, Auricularia, Cordyceps, Coriolus, Ganoderma, Grifola, Hericuim, Lentinus, Pleurotus, Polyporus, Poria, Trametes and Tremella. Non-limiting examples of a mushroom include, Agaricus augustus, Agaricus blazei, Agaricus subrufescens, Cordyceps sinensis, Coriolus versicolor, Gandoderma lucidum, Ganoderma curtisii, Gandoderma japonicum, Ganoderma oregonense, Ganoderma sinense, Ganoderma tsugae, Grifola frondosa, Grifola umbellata, Polyporus frondosus, Polyporus umbellatus, Hericuim erimaceum; Lentinus edodes, Pleurotus ostreaus, Tremella fuciformis and Trametes versicolor.

As used herein, the term “negative energy balance” refers to a condition that the body's energy expenditure exceeds its energy intake. The present invention does not specify a method and/or a regime that leads to a negative energy balance. It is noted, however, that this is generally achieved by a well-known thesis: “eat less and exercise more.” The present invention works with any regular meals and imposes no dietary restrictions though it is noted that the present invention is compatible with many diet programs that may restrict certain foods and calories. A negative energy balance may also be achieved through the use of bulking agents which bring about a feeling of satiety and appetite suppressants which act to suppress appetite; both thereby help to achieve reduced calorie intake. Additionally, a negative energy balance may be achieved by and through the compositions of the present invention which act to suppress hunger and thereby help to achieve reduced calorie intake.

As used herein, the term “nutraceutically or pharmaceutically acceptable carrier or adjuvant” refers to a carrier or adjuvant that may be administered to a subject, together with one or more liver protecting agents and one or more mushroom powder or extract of the present invention, and which does not destroy the pharmacological activity thereof and is nontoxic when administered in doses sufficient to deliver a therapeutic amount of the compound.

As used herein, the term “safe and effective” refers to a state of being that is, produces, or is capable of producing a predictable, reproducible, desired response of subject to compositions and methods herein, and which are unlikely to produce adverse side effects and/or produces only minimal/minor side effects.

As used herein, the term “substantially free” refers to a state of being wholly or almost wholly absent or lacking of a particular characteristic, e.g., a weight reduction plateau.

As used herein, the term “substantially together” refers to administering to a subject active ingredients in separate dosage forms, such that, they are administered either simultaneously or within a period of time such that the subject receives benefit of the aggregate effects of the separate dosage forms, e.g., within at least about ¼ to 3 hours of each other.

As used herein, the term “sustained weight reduction” or “sustained weight loss” refers to weight reduction or weight loss that continues substantially free from interruption (e.g., substantially free of a weight loss plateau) and that can be maintained once a target weight is reached.

As used herein, the term “weight loss plateau” refers to a state where there is little or no change and/or loss in body weight observed despite continued efforts to reduce body weight.

In a preferred embodiment, a liver protecting agent is selected on a basis of the therapeutic protection and/or treatment of the liver: in particular, the ability to rejuvenate the fatty liver.

Milk thistle (Silybum marianum), a plant native to the Mediterranean, is also known by the names, blessed milk thistle, St. Mary thistle, Cardui mariae fructus, and silymarin. Milk thistle extract provides hepatocellular protection by stabilizing hepatic cell membranes. McPartland, J. M. (1996) Complimentary Medicine International 3(2):40-42. Other actions of hepatoprotection include interruption of enterohepatic recirculation of toxins, stimulation of protein synthesis and regeneration of damaged hepatocytes, as well as, antioxidant activity. McPartland, 1996.

Preferably milk thistle is in extract form. Milk thistle extract is prepared from the fruits (seeds) of Silybum marianum and usually standardized to a concentration of 70% to 80% of three flavonolignans (silibinin, silychristin and silydianin, collectively known as silymarin) needed for therapeutic and clinical liver protection. German Commission E Monographs: Therapeutic Guide to Herbal Medicines. Milk thistle is safe, having no known contraindications and only a mild laxative side effect in occasional instances. German Commission E Monographs.

Schisandra (Schisandra chinensis) has been a primary medicinal agent of Chinese herbal medicine for the treatment of liver ailments since antiquity and recent research has confirmed its hepatoprotective effects in treating liver damages such as hepatitis. American Herbal Pharmacopoeia and Therapeutic Compendium, Upton, Roy (Ed.) (1999). Schisandra is also known by the names Schizandra, Schisandra chinensis, Wu wei zi, bei wu wei zi, ngu mei gee, m mei gee, Chinesischer Limonenbaum, gomishi, chosen-gomischi, matsbouza, omicha, bac ngu vi tu and Limonnik kitajskij.

Schisandra is generally prepared in a decoction or powder formulation from the dried fruits. Recommended doses of the decoction formulation are about 1.5 g to 10 g daily. More preferably, schisandra is prepared as a powder formulation. Recommended doses of powder formulations are about 1.5 g to 10 g daily based on guidelines in the Pharmacopoeia of the People's Republic of China 1997. Schisandra has a relatively safe profile having no toxicity within the disclosed doses and minor reported side effects such as heartburn, acid indigestion, stomach pain, anorexia, allergic skin rashes and urticaria upon indigestion of large doses. American Herbal Pharmacopoeia and Therapeutic Compendium, Upton, Roy (Ed.) October 1999.

Fruits of Lycium (Lycium barbarum), commonly known as the wolfberry, produce a sweet, tonic decoction that has been used in Asian countries since antiquity to lower blood pressure and cholesterol levels, acting mainly on the liver and kidney. Lycium is known as matrimony vine, Lycium europaeum, and Lycium chinense. In a preferred embodiment of the present invention, lycium fruits are used as a form of decoction or dried fruits are made into a powder formulation.

Other liver protecting agents useful in the invention include Andrographis, artichoke, Artemisia, astragalus, barberry, boldo, bupleurum, dandelion, dong quai, fo-ti, fringe tree, fumitory, gotu kola, guggul, kudzu, licorice, neem, Phyllanthus, Picrorrhiza, prickly pear, rehmannia, skullcap, and tumeric. These liver protecting agents can be used in powder form, as an extract, or derivative thereof.

In still another preferred embodiment, a mushroom powder, extract, or derivatives thereof, are selected on a basis of the hunger suppressing activity and fat burning capability when used in combination with the liver protective agent. More preferably, a mushroom is selected from a group of genus consisting of Agaricus, Auricularia, Cordyceps, Coriolus, Ganoderma, Grifola, Hericuim, Lentinus, Pleurotus, Polyporus, Poria, Trametes and Tremella. Most preferably, the mushroom is selected from Agaricus augustus, Agaricus blazei, Agaricus subrufescens (the “agaricus”); Cordyceps sinensis (the “cordyceps”); Coriolus versicolor (the “coriolus”); Gandoderma lucidum, Ganoderma curtisii, Gandoderma japonicum, Ganoderma oregonense, Ganoderma sinense, and Ganoderma tsugae (the “reishi”); Grifola frondosa, Grifola umbellata, Polyporus frondosus, Polyporus umbellatus, (the “maitake”); Hericuim erimaceum (the “Yamabushitake”); Lentinus edodes (the “shiitake”), Pleurotus ostreaus (the “oyster mushroom”), Tremella fuciformis and Trametes versicolor. The mushroom of the present invention is preferably formulated into a powder formulation or as an extract.

The mushrooms listed above are known as medicinal mushrooms being beneficial in stimulating the immune system; lowering cholesterol and blood glucose levels acting on the liver and kidney; being hepatoprotective; and others. It is also shown that Lentinus edodes, Grifola frondosa, or Ganoderma lucidum partially inhibits fat absorption in the gastro-intestine. The noble features of the present invention, hunger suppression and fat burning properties, appear to originate from all combined actions of the mushrooms and hepatoprotective milk thistle, schisandra, and/or lycium. The mushrooms of the present invention are food products and the maximum amounts used in the present invention are generally less than the amounts that are safely consumed as foods. American Herbal Pharmacopoeia and Therapeutic Compendium. Growing Gourmet and Medicinal Mushrooms by Paul Stamets (2000).

Moreover, the mushrooms of the present invention are known throughout the world by many different cultural and common names. Exemplary names of Agaricus (Agaricus blazei) are Royal Sun Agaricus, Himematsutake, Kawaariharatake, Cogmelo de Deus (mushroom of God) Murrill's Agaricus or ABM, King Agaricus, Almond Portobello. Reishi (Ganoderma lucidum and Ganoderma japonicum) is also known in China as Ling Zhi, Ling Zhi Cao, Ling Chih, Hong Ling Zhi, Chi Zhi, He Ling Zhi, and Zi Zhi which is Chinese for “Tree of Life”. In Japan reishi (Japanese for “Divine or Spiritual Mushroom”) is also called Mannentake (for “10,000-year Mushroom” or “Mushroom of Immortality”), Saiwai-take (for “Good-fortune Mushroom”), Sarunouchitake (for “Monkey's Seat”) and Rokkaku. In Korea reishi is called Young ji, and in Vietnam named Ling chi. Also, maitake (Grifola frondosa or Grifola umbellata) is known by other names in different countries, such as, Hen-of-the-Woods, Dancing butterfly Mushroom, Chorei-maitake, Kumotake (the “Cloud Mushroom) and Tsuchi-maitake in Japan, and Zhu Ling and Chinese Sclerotium in China.

The dietary supplement compositions disclosed herein comprising at least one liver protecting agent and at least one mushroom can also be combined with a bulking agent. Bulking agents swell in the stomach and create a feeling of fullness. Non-limiting examples of bulking agents include natural fibers such as, bran, cellulose, chitin, chitosan, glucomannan, microcrystalline cellulose, hemicellulose, husk, kelp, lignin, psyllium; and gums, such as tragacanth, guar gum, carrageenan, glucomannanes and alginates.

Further, the dietary supplement compositions disclosed herein may further comprise one or more nutraceutically or pharmaceutically acceptable carrier or adjuvant. Such carriers or adjuvants that may be used in the dietary supplement compositions of the present invention include, but are not limited to, ion exchangers, alumina, aluminum stearate, lecithin, self-emulsifying drug delivery systems (SEDDS) such as d-alpha-tocopherol polyethyleneglycol 1000 succinate, or other similar polymeric delivery matrices or systems, serum proteins, such as human serum albumin, buffer substances such as phosphates, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium chloride, zinc salts, colloidal silica, magnesium trisilicate, polyvinyl pyrrolidone, cellulose-based substances, polyethylene glycol, sodium carboxymethylcellulose, polyacrylates, waxes, polyethylene-polyoxypropylene-block polymers, polyethylene glycol and wool fat. Cyclodextrins such as alpha-, beta-, and gamma-cyclodextrin, or chemically modified derivatives such as hydroxyalkylcyclodextrins, including 2- and 3-hydroxypropyl-.beta.-cyclodextrins, or other solublized derivatives may also be advantageously used to enhance delivery of weight-reducing effective liver protecting agents and mushroom powders or extracts of the present invention.

The dietary supplement compositions of the present invention may be formulated into liquid, suspension or solid dosage forms or combinations thereof. For example, the dietary supplement compositions may be taken or administered as a single unitary dose containing both a liver protecting agent(s) and a mushroom powder, in a liquid, suspension or solid dosage from. Likewise, the present invention contemplates taking the ingredients substantially together, but separately in the same or different dosage forms, such as, taking a liver protecting agent as a liquid dose, a mushroom as a solid dose, as a suspension dose or combinations thereof, or taking or administering them separately as either solid, liquid, or suspension doses. It is preferable to formulate each of the active ingredients into solid dosage forms such as tablets or capsules. Most preferably, a single unitary dose which comprises all of the active ingredients is formulated into a solid dosage form such as a capsule or tablet. Most preferably, a dosage form comprises the daily dosage of all active ingredients.

When administering or taking the active ingredients substantially together, but separately in same or different dosage forms, the order in which they are ingested is not critical. In other words, a liver protecting agent and a mushroom may be ingested simultaneously, or the liver protecting agent may be ingested first followed by the mushroom, or the mushroom may be first ingested followed by the liver protecting agent. It is preferable to formulate at least one liver protecting agent and at least one mushroom powder or extract into a solid dosage form, such as a tablet or capsule, which can be co-ingested substantially together. Most preferably, a single unitary dose which comprises all of the active ingredients is formulated to be co-ingested in a solid dosage form such as a capsule or tablet.

The dietary supplement compositions of the present invention can be conveniently prepared from, for example, commercially available liver protecting agents and mushroom formulations. Non-limiting examples of commercially available liver protecting agent formulations include, AMNI® (Advanced Medical Nutrition, Inc.), NATURE'S ANSWER® (Bio-Botanica, Inc.), NATURE'S HERBS® (Nature's Herbs, Inc.), SOLARAY® (Solaray, Inc.), SOLGAR® (American Home Products Corporation) and ZAND® (Batonical Laboratories, Inc.). Non-limiting examples of commercially available mushroom formulations include, MAITAKE GOLD® (Tradeworks Group, Inc.), MUSHROOMSCIENCE® (JHS Natural Products LLC), PLANETARY FORMULAS® (Threshold Enterprises Ltd.), REISHIMAX® (Pharmanex, LLC) (these mushroom formulations are prepared primarily as immune stimulants). However, tests whether the quality and content of these commercial products are good for the management and control of body weight in accordance with the present invention have not been performed.

As will be appreciated, the compositions of the present invention comprise a safe and effective dietary supplement composition comprising at least one liver protecting agent, such as milk thistle, schisandra, lycium, or derivatives thereof, and at least one mushroom powder, extract, or derivatives thereof, and one or more nutraceutically or pharmaceutically acceptable carrier in effective doses to provide sustained weight loss. Likewise, the methods of the present invention comprise administering a composition or compositions of the invention, wherein the active ingredients are administered and ingested in safe and effective doses to provide sustained weight loss. Preferably, the compositions are administered in combination with a method and/or regimen that attains a negative energy balance.

Weight-reducing effective amounts of the present compositions include a daily dosage of at least one liver protecting agent and at least one mushroom powder or extract in a weight ratio varying from 1:100 to 100:1, respectively. Most preferably, the effective amounts of the present compositions include a daily dosage of at least one liver protecting agent and at least one mushroom powder or extract in a weight ratio of about 1:1.

In one embodiment of the invention, the compositions of the present invention comprise one, two or three liver protecting agents present in a combined total daily dose of about 0.01 g (10 mg) to 20 g, regardless of whether one, two or three liver protecting agents are present in a single unitary dosage form or separate dosage form and regardless of whether they are taken together or substantially together. Preferably, the compositions of this embodiment of the invention comprise the liver protecting agents milk thistle, schisandra and/or lycium. Milk thistle extract is preferably present in a daily amount ranging from about 0.01 g (10 mg) to about 10 g. Milk thistle, schisandra and/or lycium dried fruit powder is preferably present in a daily amount ranging from about 0.01 g (10 mg) to 20 g.

In another embodiment of the invention, the compositions of the present invention comprise one or more mushrooms present in a combined total daily dose of about 0.01 g (10 mg) to 20 g, regardless of whether one or more mushrooms are present in a single unitary dosage form or separate dosage form and regardless of whether they are taken together or substantially together.

It is further preferred that the daily dosage of the compositions of the present invention is administered or ingested in part over the course of a day. More specifically, the daily dosage is administered or ingested in one or more, e.g., one to six, or preferably one to four divided approximately equal amounts at different times during the day. Most preferably, the daily dosage is administered or ingested in three divided approximately equal amounts at different times during the day. Preferably, each of the approximately equal amounts is administered or ingested within about two hours before eating, such as before each daily meal (daily meals consisting of breakfast, lunch and dinner) to about 30 minutes after each daily meal. Most preferably, each of the approximately equal daily doses is administered or ingested within about 30 minutes before a meal.

Administration of the compositions under this protocol is useful for lowering cholesterol levels, reducing glucose levels to treat diabetes, and lowering blood pressure to treat hypertension. Administration of the compositions is useful for healing a liver damaged by alcohol, drugs, or chemicals.

The dietary supplement compositions may be in a form suitable for oral use, for example, as tablets, lozenges, aqueous or oily suspensions, dispersible powders or granules, emulsions, hard or soft capsules, syrups, decoction, elixirs, herb teas, vials or drops. Compositions intended for oral use may be prepared according to any method known to the art for the manufacturer of pharmaceutical or nutraceutical compositions and such compositions may contain one or more agents such as, for example, sweetening agents, flavoring agents, coloring agents and the like, in order to provide a pharmaceutically elegant and palatable preparation.

Tablets contain the active ingredients in admixture with nontoxic nutraceutically or pharmaceutically acceptable excipients which are suitable for manufacture of tablets. These excipients may be inert diluents, for example, calcium carbonate, sodium carbonate, lactose, calcium phosphate or sodium phosphate; granulating and disintegrating agents, for example, alginic acid, croscarmellose sodium, maize starch or; binding agents, for example, acacia, gelatine or starch, and lubricating agents, for example, magnesium stearate or stearic acid. The tablets may be uncoated or they may be coated by known techniques to delay disintegration and absorption in the gastro-intestinal tract and thereby provide an even longer sustained action over a period of time. The tablets may be chewable or non-chewable and designed to desired weight, potency and hardness through well-known methods in the pharmaceutical arts.

Formulations for oral use may also be presented as hard gelatin capsules wherein the active ingredients are mixed with an inert solid diluent, for example, calcium carbonate, calcium phosphate or kaolin, or as soft gelatin capsules wherein the active ingredient is mixed with a suitable oil medium, for example, arachis oil, liquid paraffin or olive oil.

Formulations for oral use may also be presented as lozenges wherein the active ingredients are mixed into a hard candy composition. Suitable hard candy compositions can be made from varying, but highly concentrated, sucrose solutions including corn syrup as a second essential ingredient. Other known hard candy compositions may utilize any suitable good testing, sweet excipient other than sucrose.

Aqueous suspensions contain the active ingredients in admixture with excipients suitable for the manufacture of aqueous suspensions. Such excipients or combinations thereof may be suitable suspending agents, for example, alginates, carboxymethylcellulose, carboxypolymethylene, carrageenan, colloidal silicon dioxide, corn starch, flowable starch, gelatin, guar gum, gum acacia, gum tragacanth, hydroxypropylcellulose, hydroxypropylmethylcellulose, maltodextrin, methylcellulose, microcrystalline cellulose, pectin, polyethylene glycol 800, polyvinyl alcohol, polyvinylpyrrolidone, sodium alginate, sodium carboxymethyl cellulose or xanthum gum; dispersing or wetting agents may be any suitable naturally occurring phosphatide, for example, lecithin, or condensation products of an alkylene oxide with fatty acids, for example, polyoxyethylene stearate, or condensation products of ethylene oxide with long chain aliphatic alcohols, for example, heptadecaethyleneoxycetanol, or condensation products of ethylene oxide with partial esters derived from fatty acids and a hexitol, for example, polyoxyethylene sorbitol monnoleate, or condensation product of ethylene oxide with partial esters derived from fatty acids and hexitol and anhydrides, for example, polyoxyethelyne sobirtan monooleate, or water. The aqueous suspensions may also contain one or more suitable preservatives, for example, ethyl, or n-propyl, p-hydroxy benzoate, one or more suitable coloring agents, one or more suitable flavoring agents such as, cinnamon, chocolate, fruit flavors (i.e., cherry, grape, orange, strawberry, etc.), menthol, mints, vanilla and combination of two or more thereof, one or more suitable sweetening agents, such as calcium cyclamate, dextrose, fructose, galactose, glucose, glycerin, maltose, mannitol, mannose, ribose, partially hydrolyzed starch solids, partially hydrolyzed corn syrup solids, sodium cyclamate, sorbital, sucralose, sucrose, xylitol, or xylose, and one or more suitable coloring agents.

Dispersible powders and granules suitable for preparation of an aqueous suspension by the addition of water provide the active ingredient in admixture with a dispersing or wetting agent, suspending agent and one or more preservatives. Suitable dispersing or wetting agents and suspending agents may be exemplified by those already mentioned above. Additional suitable excipients, for example, sweetening, flavoring and coloring agents, may also be present.

Syrups and elixirs may be formulated with suitable sweetening agents, for example, one or more of glycerol, sorbitol, sucrose or xylose. Such formulations may also contain suitable demulcents, preservatives and flavoring and coloring agents.

Moreover, an effective amount of each component of the present compositions varies according to the specific combination of components, the derivative forms of the components and inter-individual differences in body weight. An effective dose may take into consideration inter-individual differences in body weight and effective doses can be adjusted according to methods known by one skilled in the art. The following examples are presented for illustrative purposes and it is to be understood that the present invention is not limited to those precise embodiments, and that various changes and modifications can be effected therein by one skilled in the art without departing from the scope and spirit of the invention as defined by the appended claims.

### EXAMPLE 1

An oral dietary supplement composition for the management and control of body weight is formulated having the following composition in the following daily dosage amount for an individual weighing about 150 lbs.

| Ingredient | Weight |
|---|---|
| Milk Thistle Extract (80% silymarin) | 500 mg |
| Schisandra Powder of dried fruits | 2000 mg |
| Maitake Powder of dried mushroom | 1500 mg |
| Reishi Powder of dried mushroom | 1500 mg |

The above dietary supplement composition is prepared by known methods in the pharmaceutical and nutraceutical arts. This formulation provides a daily dosage of the active ingredients to be divided into approximately three equal amounts to be taken three times a day. For example, if a daily dose for a person weighing about 150 lbs. consists of about three capsules or tablets taken three times a day, then, a person weighing about 200 lbs. takes about four capsules three times a day.

### EXAMPLE 2

An oral dietary supplement composition for the management and control of body weight is formulated having the following composition in the following daily dosage amount for a person weighing about 150 lbs.

| Ingredient | Weight |
|---|---|
| Milk Thistle Extract (80% silymarin) | 500 mg |
| Schisandra Powder of dried fruits | 4000 mg |
| Reishi Powder of dried mushroom | 1000 mg |
| Maitake Powder of dried mushroom | 1000 mg |
| Agaricus Powder of dried mushroom | 1000 mg |

The above dietary supplement composition is prepared by known methods in the pharmaceutical and nutraceutical arts. This formulation provides a daily dosage of the active ingredients to be divided into approximately three equal amounts to be taken three times a day. For example, if a daily dose for a person weighing about 150 lbs. consists of about three capsules or tablets taken three times a day, then, a person weighing about 200 lbs. takes about four capsules three times a day.

### EXAMPLE 3

An oral dietary supplement composition for the management and control of body weight is formulated having the following composition in the following daily dosage amount for a person weighing about 150 lbs.

| Ingredient | Weight |
|---|---|
| Lycium Extract or Powder of dried fruits | 3000 mg |
| Reishi Powder of dried mushroom | 3000 mg |

The above dietary supplement composition is prepared by known methods in the pharmaceutical and nutraceutical arts. This formulation provides a daily dosage of the active ingredients to be divided into approximately three equal amounts to be taken three times a day. For example, if a daily dose for a person weighing about 150 lbs. consists of about three capsules or tablets taken three times a day, then, a person weighing about 200 lbs. takes about four capsules three times a day.

### EXAMPLE 4

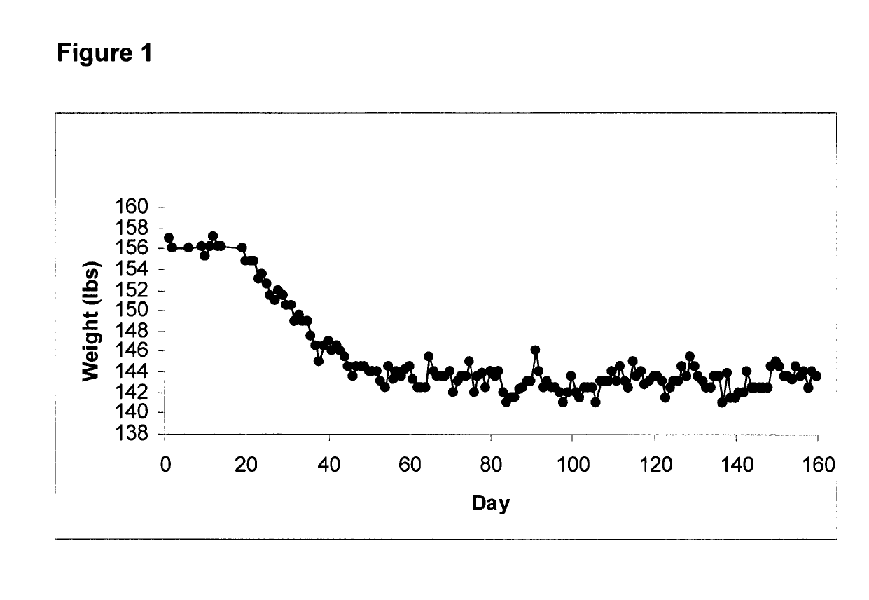

At the start of the trial an individual with about 17% body fat content (Case 1) as measured by bioelectrical impedance analysis (BIA) ate three regular meals. The individual ingested daily doses of milk thistle, schisandra, maitake, and reishi as specified in the above Example 1. After about four days from the commencement of the trial, the individual experienced suppressed feelings of hunger. As a result, the individual was able to skip lunch without feelings of hunger. Also, the individual combined exercise while eating two regular meals a day. The individual lost 12 lbs. over the course of 30-days. The individual mostly lost fat (about 9.5 lbs.), having a body fat content of about 12% as measured by BIA. Data are set forth in FIG. 1. Upon reaching the target weight, the weight loss was sustained from about day 42 and thereafter as evidenced in the data of FIG. 1.

### EXAMPLE 5

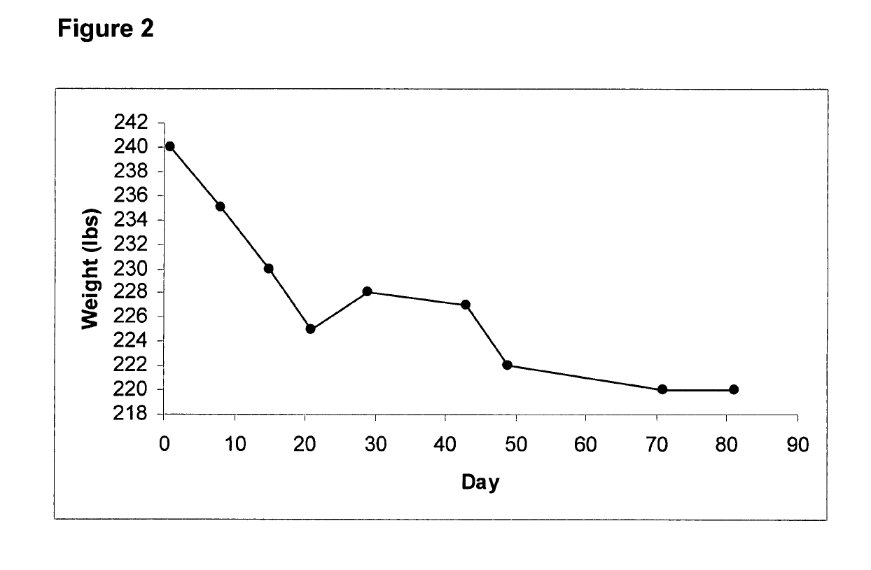

In a second case, another individual weighing about 240 lbs. (Case 2), who was taking LIPITOR® because of high cholesterol, ingested daily doses of milk thistle, schisandra, maitake and reishi as specified in Example 1. Over the course of a 10-week period, the individual lost 20 lbs. and was able to stop taking LIPITOR® due to a simultaneous reduction in that individual's cholesterol level. Data are set forth in FIG. 2. Although it appears that there was a weight loss plateau, the apparent plateau represents an interval when the individual took a break from the weight loss regime. Weight loss immediately resumed when the individual resumed the weight loss regimen.

### EXAMPLE 6

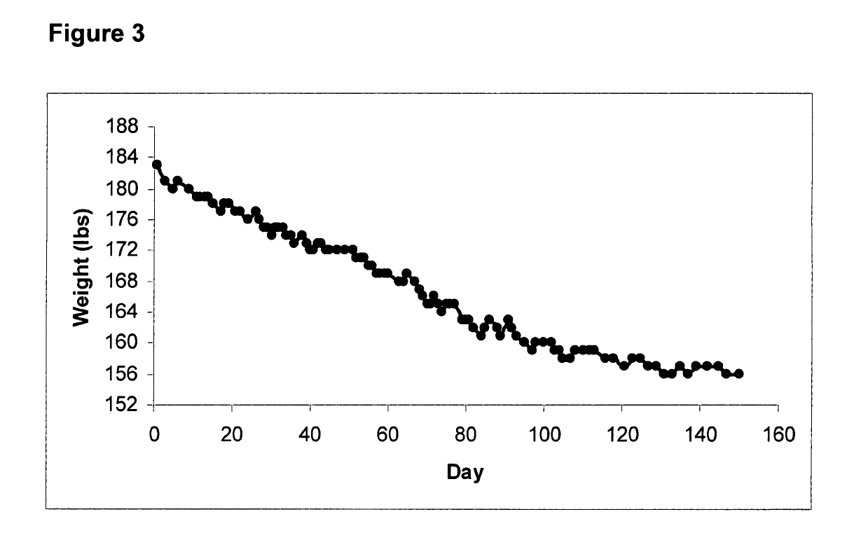

In a third case, another individual weighing about 183 lbs. (Case 3), who was taking ZOCOR® (Merck & Co., Inc.) for high cholesterol, ingested daily doses of milk thistle, schisandra, maitake, reishi and agaricus as specified in Example 2. Over the course of about 4 months, the individual lost 30 lbs. and was able to stop taking ZOCOR® due to a simultaneous reduction in that individual's cholesterol level. Data are set forth in FIG. 3.

### EXAMPLE 7

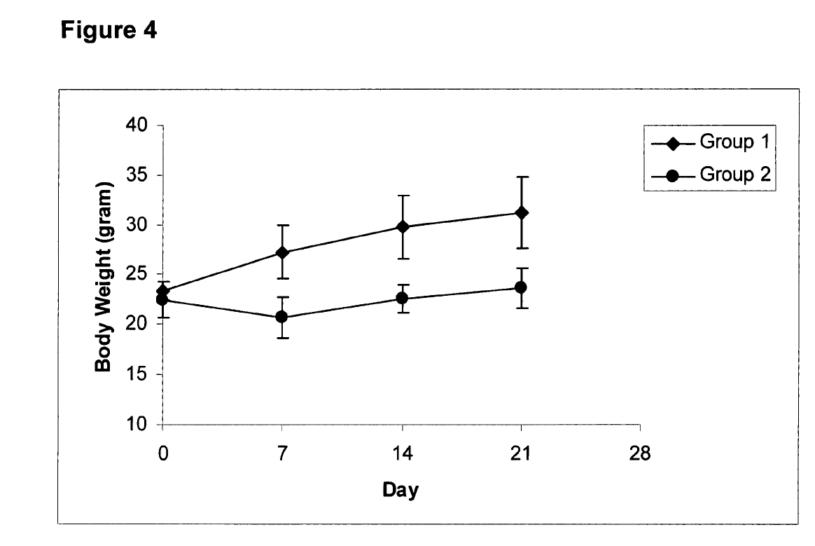

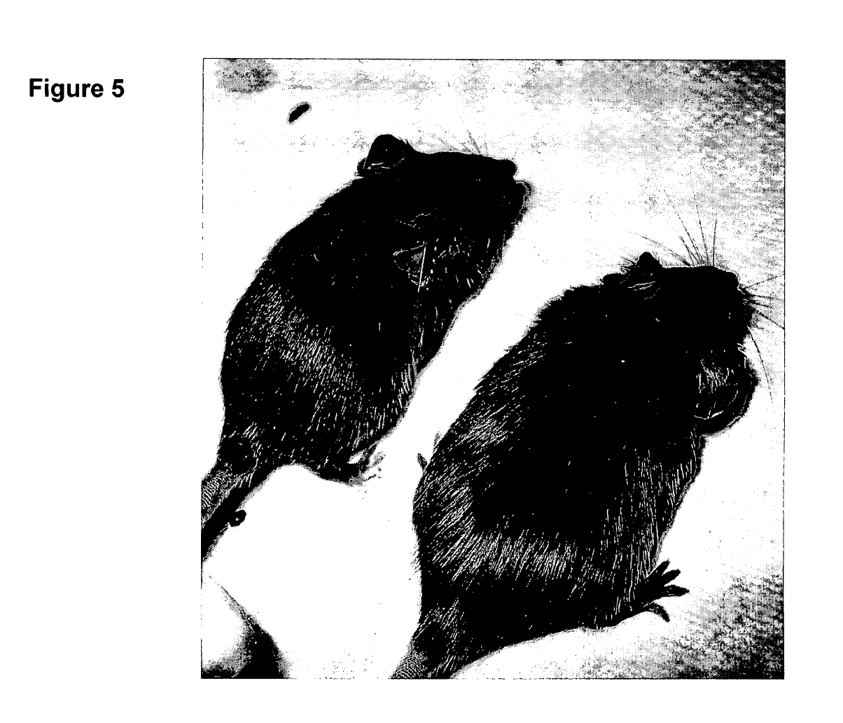

Two groups (Groups 1 and 2) of mice were fed a high fat diet, including, a 45% fat content from lard, carbohydrates and protein. The mice in Group 1, fed only the high fat diet, are the control group. The mice in Group 2 were fed the high fat diet further containing 2.5% shisandra, 2.5% lycium, 7.5% maitake and 7.5% reishi (all in powder form) blended therein. While the mice in Group 2 consumed as much or more food than the mice in Group 1, the mice in Group 2 barely gained any weight. In contrast, the mice in Group 1 gained measurable and observable weight. (FIGS. 4 and 5).

Although illustrative embodiments of the present invention have been described in detail, it is to be understood that the present invention is not limited to those precise embodiments, and that various changes and modifications can be effected therein by one skilled in the art without departing from the scope and spirit of the invention as defined by the appended claims.

1-57. (canceled)

58. A dietary supplement comprising a weight-reducing effective amount of:
at least one naturally occurring liver protecting agent powder or extract selected from the group consisting of fo-ti, lyceum, milk thistle, schisandra, and combinations thereof;
and at least one mushroom powder or extract selected from a member of the genus consisting of Agaricus, Ganoderma, Grifola, Lentinus, Trametes, and combinations thereof.

59. The dietary supplement according to claim 58, wherein the weight ratio of the liver protecting agent to the at least one mushroom ranges from about 1:100 to about 100:1.

60. The dietary supplement according to claim 58, wherein the powder or extract of the liver protecting agent is present in an amount from about 0.01 g to about 20 g.

61. The dietary supplement according to claim 58, wherein the powder or extract of mushroom is present in an amount from about 0.01 g to about 20 g.

62. The dietary supplement according to claim 58 comprising lyceum, schisandra, maitake and reishi.

63. The dietary supplement according to claim 62, wherein the powder or extract of lyceum is present in an amount from about 0.01 g to about 20 g.

64. The dietary supplement according to claim 63, wherein the powder or extract of lyceum is present in an amount from about 100 mg to about 1000 mg.

65. The dietary supplement according to claim 58, wherein the powder or extract of schisandra is present in the amount from about 0.01 g to about 20 g.

66. The dietary supplement according to claim 65, wherein the powder or extract of schisandra is present in the amount from about 100 mg to about 1000 mg.

67. The dietary supplement according to claim 62, wherein the powder or extract of maitake is present in an amount from about 0.01 g to about 20 g.

68. The dietary supplement according to claim 67, wherein a powder or extract of maitake is present in the amount from about 100 mg to about 1000 mg.

69. The dietary supplement according to claim 62, wherein the powder or extract of reishi is present in an amount from about 0.01 g to about 20 g.

70. The dietary supplement according to claim 69, wherein the powder or extract of reishi is present in the amount from about 100 mg to about 1000 mg.

71. The dietary supplement according to claim 58 comprising milk thistle, schisandra, maitake and reishi.

72. The dietary supplement according to claim 58 comprising milk thistle, schisandra, agaricus, maitake and reishi.

73. The dietary supplement according to claim 58 comprising lyceum and reishi.

74. The dietary supplement according to claim 58 comprising schisandra, maitake and reishi.

75. The dietary supplement according to claim 58 comprising fo-ti, shiitake and agaricus.

76. The dietary supplement according to claim 58 comprising lyceum, milk thistle and tremetes.

77. The dietary supplement according to claim 58 wherein the supplement is a single composition comprising the liver protecting agent, at least one mushroom and further comprising a nutraceutically acceptable carrier.

78. The dietary supplement according to claim 58 wherein the supplement is a liquid, suspension or solid dosage form.

79. The dietary supplement according to claim 76, wherein the supplement is in the form of a tablet, capsule, lozenge, granulates, syrup, tea, or liquid suspension.

80. The dietary supplement according to claim 77, wherein the dosage form is a capsule.

81. The dietary supplement according to claim 58 further comprising a bulking agent.

82. The dietary supplement according to claim 58 further comprising an appetite suppressant.

83. A method for sustained weight reduction, comprising administering to a subject in need thereof, a weight-reducing effective amount of the dietary supplement of claim 58.
